SEQUENCE LISTING (39 sequences)

SEQ ID NO: 1 - protein, 13 aa, Artificial Sequence
  Peptide
  MISC_FEATURE (1)..(1): X is V, F, I, L, or C
  MISC_FEATURE (2)..(2): X is V, I, or L
  MISC_FEATURE (3)..(3): X is G or S
  MISC_FEATURE (4)..(4): X is L, M, or I
  MISC_FEATURE (5)..(5): X is A, G, I, M, V, or L
  MISC_FEATURE (7)..(7): X is I or M
  MISC_FEATURE (8)..(8): X is V, I, C, or G
  MISC_FEATURE (9)..(9): X is A, S, V, F, C, or T
  MISC_FEATURE (11)..(11): X is V or I
  MISC_FEATURE (12)..(12): X is V, L, or I
  MISC_FEATURE (13)..(13): X is V, T, L, or I
XXXXXLXXXGXXX

SEQ ID NO: 2 - protein, 13 aa, Artificial Sequence
  Peptide
VVGLALIVAGVVV

SEQ ID NO: 3 - protein, 13 aa, Artificial Sequence
  Peptide
VVGLALINAGVVV

SEQ ID NO: 4 - protein, 13 aa, Artificial Sequence
  Peptide
FVGMGLIVSGVVV

SEQ ID NO: 5 - protein, 13 aa, Artificial Sequence
  Peptide
VVGIGLIVVGVVT

SEQ ID NO: 6 - protein, 13 aa, Artificial Sequence
  Peptide
IISIILIIFGVVL

SEQ ID NO: 7 - protein, 13 aa, Artificial Sequence
  Peptide
LLGIGLIIAGVLV

SEQ ID NO: 8 - protein, 13 aa, Artificial Sequence
  Peptide
CIGLALMIAGIVI

SEQ ID NO: 9 - protein, 13 aa, Artificial Sequence
  Peptide
IIGMMLICAGVLV

SEQ ID NO: 10 - protein, 13 aa, Artificial Sequence
  Peptide
IVSIVLIIVGVVL

SEQ ID NO: 11 - protein, 13 aa, Artificial Sequence
  Peptide
IIGMLLIICGVIV

SEQ ID NO: 12 - protein, 13 aa, Artificial Sequence
  Peptide
IIGMMLICTGVLV

SEQ ID NO: 13 - protein, 13 aa, Artificial Sequence
  Peptide
IIGIGLIIAGVVV

SEQ ID NO: 14 - protein, 27 aa, Artificial Sequence
  Peptide
NQAPSLYAISLIVVFLCLAALYESWSI

SEQ ID NO: 15 - protein, 22 aa, Artificial Sequence
  Peptide
PIFAWVIAIIIMLAGGLAILKL

SEQ ID NO: 16 - protein, 13 aa, Artificial Sequence
  Peptide
IIGIALIIAGVVV

SEQ ID NO: 17 - protein, 13 aa, Artificial Sequence
  Peptide
IIGIALINAGVVV

SEQ ID NO: 18 - protein, 13 aa, Artificial Sequence
  Peptide
IIGIALINAGVNV

SEQ ID NO: 19 - protein, 13 aa, Artificial Sequence
  Peptide
NIGIALINAGVVV

SEQ ID NO: 20 - protein, 16 aa, Artificial Sequence
  Peptide
IIGIALINAGVVVKKK

SEQ ID NO: 21 - protein, 20 aa, Artificial Sequence
  Peptide
  MOD_RES (1)..(1): ACETYLATION
  MOD_RES (2)..(4): MeGly
AXXXLLGIGLIIAGVLVKKK

SEQ ID NO: 22 - protein, 20 aa, Artificial Sequence
  Peptide
  MOD_RES (1)..(1): ACETYLATION
  MOD_RES (2)..(4): MeGly
AXXXLLVLAGIGIIGLVKKK

SEQ ID NO: 23 - protein, 28 aa, Artificial Sequence
  Peptide
  MOD_RES (1)..(1): ACETYLATION
  MOD_RES (2)..(4): MeGly
AXXXVAGVVGLALIVAGVVVLNVASKKK

SEQ ID NO: 24 - protein, 9 aa, Artificial Sequence
  Peptide
GLALIVAGV

SEQ ID NO: 25 - protein, 21 aa, Artificial Sequence
  Peptide
VAGVVGLALIVAGVVVLNVAS

SEQ ID NO: 26 - protein, 21 aa, Artificial Sequence
  Peptide
AWGFVGMGLIVSGVVVLNLLS

SEQ ID NO: 27 - protein, 21 aa, Artificial Sequence
  Peptide
VMKVVGIGLIVVGVVTLNLAG

SEQ ID NO: 28 - protein, 21 aa, Artificial Sequence
  Peptide
LISIISIILIIFGVVLLNTFG

SEQ ID NO: 29 - protein, 21 aa, Artificial Sequence
  Peptide
PAALLGIGLIIAGVLVIQLFS

SEQ ID NO: 30 - protein, 21 aa, Artificial Sequence
  Peptide
LAACIGLALMIAGIVIINVFS

SEQ ID NO: 31 - protein, 21 aa, Artificial Sequence
  Peptide
LPAIIGMMLICAGVLVINLLS

SEQ ID NO: 32 - protein, 21 aa, Artificial Sequence
  Peptide
LITIVSIVLIIVGVVLLNIFG

SEQ ID NO: 33 - protein, 21 aa, Artificial Sequence
  Peptide
MPAIIGMLLIICGVIVINLFS

SEQ ID NO: 34 - protein, 21 aa, Artificial Sequence
  Peptide
LPAIIGMMLICTGVLVINLLS

SEQ ID NO: 35 - protein, 21 aa, Artificial Sequence
  Peptide
LAAIIGIGLIIAGVVVLNLFS

SEQ ID NO: 36 - protein, 5 aa, Artificial Sequence
  Peptide
AWVIA

SEQ ID NO: 37 - protein, 5 aa, Artificial Sequence
  Peptide
AGGLA

SEQ ID NO: 38 - protein, 8 aa, Artificial Sequence
  Peptide
SLIVVFLC

SEQ ID NO: 39 - protein, 8 aa, Artificial Sequence
  Peptide
  MISC_FEATURE (2)..(3): X is any amino acid
  MISC_FEATURE (6)..(7): X is any amino acid
CXXALXXS

(19) United States
(12) Patent Application Publication
(10) Pub. No.: US 2019/0352339 A1
(43) Pub. Date: Nov. 21, 2019

(54) PEPTIDE INHIBITOR OF TRANSMEMBRANE PORE FORMATION AND EFFLUXPUMP FUNCTION IN A SMALL MULTIDRUG RESISTANCE PROTEIN FROM PSEUDOMONAS AERUGINOSA

(71) Applicant: The Hospital for Sick Children, Toronto (CA)

(72) Inventor: Charles M. Deber, Toronto (CA)

(21) Appl. No.: 16/479,443

(22) PCT Filed: Jan. 19, 2018
(86) PCT No.: PCT/CA2018/050064

§ 371 Date: Jul. 19, 2019

Related U.S. Application Data
(60) Provisional application No. 62/449,066, filed on Jan. 22, 2017.

(51) Int. Cl. C07K 7/08; C07K 14/21; A61P 31/04

(52) U.S. Cl. CPC C07K 7/08; A61K 38/00; A61P 31/04; C07K 14/21

(57) ABSTRACT

A peptide comprises the sequence: X₁-X₂-(G or S)-X₃-X₄-L-(I or M)-X₅-X₆-G-(V or I)-X₇X₈ wherein each of X₁-X₈ is independently any amino acid and is independently present or absent; wherein the peptide has fewer than 30 amino acid residues; and wherein the peptide binds to and inhibits an efflux pump, with the proviso that the peptide does not comprise VVGLALIVAGVVV or VVGLALINAGVVV

### FIELD

The present invention relates to peptides. More specifically, the present invention is, in aspects, concerned with peptides and methods for inhibiting multimeric membrane-embedded proteins.

### BACKGROUND

Bacterial multidrug resistance (MDR) is the biggest crisis in infectious diseases. Yet there is a lack of new antibiotics, and the last line of defense is being reached for ways to make existing drugs work better. The major cause of MDR is the ability of bacteria to extrude antimicrobial molecules via membrane-embedded efflux transporters [Kourtesi, C. et al., 2013, Open Microbiol. J. 7, 34-35]. These protein pumps mediate resistance by decreasing the intracellular concentration of toxic chemicals, and most accommodate a broad spectrum of substrates. Among several families of such pumps, the small MDR proteins (SMRs) provide an accessible system for study: as pumps of only ˜110 residues folded into four transmembrane (TM) helices, SMR monomers are physically too small to extrude molecules the size of common antibiotics, and therefore must assemble into dimers to create a substrate pathway [Schuldiner, S., 2009, Biochim. Biophys. Acta—Proteins Proteomics 1794, 748-762].

Poulsen, B. E., et al. (2009, J. Biol. Chem. 284, 9870-9875 and 2011, J. Bacteriology 193, 5929-5935) describe studies in which the SMR dimerization interface was localized to a seven-residue motif on the fourth helical segment (TM4).

Poulsen, B. E. and Deber, C. M. (2012, Antimicrob. Agents Chemother. 56, 3911-3916) and Bellmann-Sickert, K., et al. (2015, J. Biol. Chem. 290, 1752-1759) describe studies in which peptides with a complementary motif that competed out these TM4-TM4 helix-helix interactions were designed, and demonstrated that the peptides successfully inhibited toxicant efflux from bacteria.

There is a need for alternative compositions to overcome or mitigate at least some of the deficiencies of the prior art, or to provide a useful alternative.

### SUMMARY

In accordance with an aspect, there is provided a peptide comprising the sequence:

X₁-X₂-(G or S)-X₃-X₄-L-(I or M)-X₅-X₆-G-(V or I)-X₇X₈

wherein each of X₁-X₈ is independently any amino acid and is independently present or absent; wherein the peptide has fewer than 30 amino acid residues; and wherein the peptide binds to and inhibits an efflux pump, with the proviso that the peptide does not comprise VVGLALIVAGVVV or VVGLALINAGVVV.

In an aspect, each of X₁-X₈ is present.

In an aspect:

X₁ is V, F, I, L, or C;

X₂ is V, I, or L;

X₃ is L, M, or I;

X₄ is A, G, I, M, V, or L;

X₅ is V, I, C, or G;

X₆ is A, S, V, F, C, or T;

X₇ is V, L, or I; and

X₈ is V, T, L, or I,

optionally wherein up to three of X₁-X₈ are independently replaced with a residue selected from N and Q so as to reduce the overall hydrophobicity of the peptide.

In an aspect, the peptide comprises a sequence selected from the group consisting of:

FVGMGLIVSGVVV;
VVGIGLIVVGVVT;
IISIILIIFGVVL;
LLGIGLIIAGVLV;
CIGLALMIAGIVI;
IIGMMLICAGVLV;
IVSIVLIIVGVVL;
IIGMLLIICGVIV;
IIGMMLICTGVLV; and
IIGIGLIIAGVVV.

In an aspect, the peptide further comprises a solubility-increasing tag.

In an aspect, the solubility-increasing tag is positively charged.

In an aspect, the solubility-increasing tag comprises one or more positively charged amino acid residues, such as from about 1 to about 10 amino acid residues, such as from about 2 to about 6 amino acid residues, such as three amino acid residues.

In an aspect, the positively charged amino acid residues are selected from lysine and/or arginine residues, such as lysine residues.

In an aspect, the solubility-increasing tag is at the C- or N-terminus of the peptide, such as the N-terminus.

In an aspect, the peptide further comprises a membrane-insertion tag.

In an aspect, the membrane-insertion tag is uncharged.

In an aspect, the membrane-insertion tag comprises one or more peptoid residues, such as from about 1 to about 10 peptoid residues, such as from about 2 to about 6 peptoid residues, such as three peptoid residues.

In an aspect, the peptoid residues are selected from NVal (N-isopropylglycine), NLeu (N-isobutylglycine), and/or NAla (N-methylglycine; sarcosine), such as sarcosine.

In an aspect, the membrane-insertion tag is at the C- or N-terminus of the peptide, such as the C-terminus.

In an aspect, the membrane-insertion tag is at the N-terminus and comprises an N-terminal amino group blocking moiety, such as an N-acetyl-Ala residue.

In an aspect, the membrane-insertion tag comprises the sequence N-acetyl-Ala-Sar-Sar-Sar-.

In an aspect, the membrane-insertion tag is at the C-terminus and comprises a C-terminal carboxylate group blocking moiety, such as a Sar-methyl ester residue.

In an aspect, the membrane-insertion tag comprises the sequence -Sar-Sar-Sar-Sar-methyl ester.

In an aspect, the peptide is stapled/macrocyclic.

In an aspect, the peptide is stapled via or between residues X₄ and X₆.

In an aspect, the peptide has a hydrophobicity above that required for insertion into a bilayer membrane but below that required for hemolysis of red blood cells.

In an aspect, the efflux pump is a bacterial drug efflux pump.

In an aspect, the efflux pump is a member of the SMR family.

In an aspect, the peptide comprises 5, 6, 7, 8, 9, 10, 11, 12, 13, 14, 15, 16, 17, 18, 19, 20, 21, 22, 23, 24, 25, 26, 27, 28, 29, or 30 amino acid residues, such as 13 amino acid residues.

In accordance with an aspect, there is provided a peptide comprising the sequence IIGIGLIIAGVVV, or a fragment or variant thereof having at least 50%, identity to IIGIGLIIAGVVV, wherein the peptide has fewer than 30 amino acid residues and wherein the peptide binds to and inhibits a drug efflux pump, with the proviso that the peptide does not comprise VVGLALIVAGVVV or VVGLALINAGVVV.

In an aspect, the peptide has at least 60%, 70%, 75%, 80%, 85%, 90%, 95%, 96%, 97%, 98%, or 99% identity to IIGIGLIIAGVVV.

In an aspect, the peptide further comprises a solubility-increasing tag.

In an aspect, the solubility-increasing tag is positively charged.

In an aspect, the solubility-increasing tag comprises one or more positively charged amino acid residues, such as from about 1 to about 10 amino acid residues, such as from about 2 to about 6 amino acid residues, such as three amino acid residues.

In an aspect, the positively charged amino acid residues are selected from lysine and/or arginine residues, such as lysine residues.

In an aspect, the solubility-increasing tag is at the C- or N-terminus of the peptide, such as the N-terminus.

In an aspect, the peptide further comprises a membrane-insertion tag.

In an aspect, the membrane-insertion tag is uncharged.

In an aspect, the membrane-insertion tag comprises one or more peptoid residues, such as from about 1 to about 10 peptoid residues, such as from about 2 to about 6 peptoid residues, such as three peptoid residues.

In an aspect, the peptoid residues are selected from NVal (N-isopropylglycine), NLeu (N-isobutylglycine), and/or NAla (N-methylglycine; sarcosine), such as sarcosine.

In an aspect, the membrane-insertion tag is at the C- or N-terminus of the peptide, such as the C-terminus.

In an aspect, the membrane-insertion tag is at the N-terminus and comprises an N-terminal amino group blocking moiety, such as an N-acetyl-Ala residue.

In an aspect, the membrane-insertion tag comprises the sequence N-acetyl-Ala-Sar-Sar-Sar-.

In an aspect, the membrane-insertion tag is at the C-terminus and comprises a C-terminal carboxylate group blocking moiety, such as a Sar-methyl ester residue.

In an aspect, the membrane-insertion tag comprises the sequence -Sar-Sar-Sar-Sar-methyl ester.

In an aspect, the peptide is stapled/macrocyclic.

In an aspect, the peptide has a hydrophobicity above that required for insertion into a bilayer membrane but below that required for hemolysis of red blood cells.

In an aspect, the efflux pump is a bacterial drug efflux pump.

In an aspect, the efflux pump is a member of the SMR family.

In an aspect, the peptide comprises 5, 6, 7, 8, 9, 10, 11, 12, 13, 14, 15, 16, 17, 18, 19, 20, 21, 22, 23, 24, 25, 26, 27, 28, 29, or 30 amino acid residues, such as 13 amino acid residues.

In accordance with an aspect, there is provided a peptide comprising the sequence NQAPSLYAISLIVVFLCLAALYESWSI, or a fragment or variant thereof having at least 50%, 60%, 70%, 75%, 80%, 85%, 90%, 95%, 96%, 97%, 98%, or 99% identity to NQAPSLYAISLIVVFLCLAALYESWSI, wherein the peptide has fewer than 30 amino acid residues and wherein the peptide binds to and inhibits a drug efflux pump.

In an aspect, the peptide binds to and inhibits interactions between TM1 and TM8 of the RND drug efflux pump.

In an aspect, the peptide further comprises a solubility-increasing tag.

In an aspect, the solubility-increasing tag is positively charged.

In an aspect, the solubility-increasing tag comprises one or more positively charged amino acid residues, such as from about 1 to about 10 amino acid residues, such as from about 2 to about 6 amino acid residues, such as three amino acid residues.

In an aspect, the positively charged amino acid residues are selected from lysine and/or arginine residues, such as lysine residues.

In an aspect, the solubility-increasing tag is at the C- or N-terminus of the peptide, such as the N-terminus.

In an aspect, the peptide further comprises a membrane-insertion tag.

In an aspect, the membrane-insertion tag is uncharged.

In an aspect, the membrane-insertion tag comprises one or more peptoid residues, such as from about 1 to about 10 peptoid residues, such as from about 2 to about 6 peptoid residues, such as three peptoid residues.

In an aspect, the peptoid residues are selected from NVal (N-isopropylglycine), NLeu (N-isobutylglycine), and/or NAla (N-methylglycine; sarcosine), such as sarcosine.

In an aspect, the membrane-insertion tag is at the C- or N-terminus of the peptide, such as the C-terminus.

In an aspect, the membrane-insertion tag is at the N-terminus and comprises an N-terminal amino group blocking moiety, such as an N-acetyl-Ala residue.

In an aspect, the membrane-insertion tag comprises the sequence N-acetyl-Ala-Sar-Sar-Sar-.

In an aspect, the membrane-insertion tag is at the C-terminus and comprises a C-terminal carboxylate group blocking moiety, such as a Sar-methyl ester residue.

In an aspect, the membrane-insertion tag comprises the sequence -Sar-Sar-Sar-Sar-methyl ester.

In an aspect, the peptide is stapled/macrocyclic.

In an aspect, the peptide has a hydrophobicity above that required for insertion into a bilayer membrane but below that required for hemolysis of red blood cells.

In an aspect, the efflux pump is a bacterial drug efflux pump.

In an aspect, the efflux pump is a member of the SMR family.

In an aspect, the peptide comprises 5, 6, 7, 8, 9, 10, 11, 12, 13, 14, 15, 16, 17, 18, 19, 20, 21, 22, 23, 24, 25, 26, 27, 28, 29, or 30 amino acid residues, such as 13 amino acid residues.

In accordance with an aspect, there is provided a peptide comprising about 5 to about 30 amino acid residues, wherein the peptide binds to and inhibits interactions between two or more transmembrane regions of a drug efflux pump, thereby inhibiting the drug efflux by the pump, with the proviso that the peptide does not comprise VVGLALIVAGVVV or VVGLALINAGVVV.

In accordance with an aspect, there is provided a peptide that competitively inhibits multimerization of a membrane-embedded protein, wherein the peptide is complementary to a motif within a helical transmembrane segment of the protein and thereby competitively inhibits a helix-helix interaction between monomers to inhibit multimerization of the protein, with the proviso that the peptide does not comprise VVGLALIVAGVVV or VVGLALINAGVVV.

In accordance with an aspect, there is provided a peptide that inhibits protein self-assembly via membrane-penetrating peptides containing topology complementary to helix-helix interaction site(s), thereby competitively binding and disrupting their assembly motif and inhibiting their efflux function, with the proviso that the peptide does not comprise VVGLALIVAGVVV or VVGLALINAGVVV.

In an aspect, the drug efflux pump is a bacterial drug efflux pump.

In an aspect, the drug efflux pump belongs to a family selected from the group consisting of SMR, RND, MFS, MATE, ABC, and combinations thereof.

In an aspect, the peptide binds to and inhibits interactions between TM1 and TM8 of the RND drug efflux pump.

In an aspect, the peptide further comprises a solubility-increasing tag.

In an aspect, the solubility-increasing tag is positively charged.

In an aspect, the solubility-increasing tag comprises one or more positively charged amino acid residues, such as from about 1 to about 10 amino acid residues, such as from about 2 to about 6 amino acid residues, such as three amino acid residues.

In an aspect, the positively charged amino acid residues are selected from lysine and/or arginine residues, such as lysine residues.

In an aspect, the solubility-increasing tag is at the C- or N-terminus of the peptide, such as the N-terminus.

In an aspect, the peptide further comprises a membrane-insertion tag.

In an aspect, the membrane-insertion tag is uncharged.

In an aspect, the membrane-insertion tag comprises one or more peptoid residues, such as from about 1 to about 10 peptoid residues, such as from about 2 to about 6 peptoid residues, such as three peptoid residues.

In an aspect, the peptoid residues are selected from NVal (N-isopropylglycine), NLeu (N-isobutylglycine), and/or NAla (N-methylglycine; sarcosine), such as sarcosine.

In an aspect, the membrane-insertion tag is at the C- or N-terminus of the peptide, such as the C-terminus.

In an aspect, the membrane-insertion tag is at the N-terminus and comprises an N-terminal amino group blocking moiety, such as an N-acetyl-Ala residue.

In an aspect, the membrane-insertion tag comprises the sequence N-acetyl-Ala-Sar-Sar-Sar-.

In an aspect, the membrane-insertion tag is at the C-terminus and comprises a C-terminal carboxylate group blocking moiety, such as a Sar-methyl ester residue.

In an aspect, the membrane-insertion tag comprises the sequence -Sar-Sar-Sar-Sar-methyl ester.

In an aspect, the peptide is stapled/macrocyclic.

In an aspect, the peptide has a hydrophobicity above that required for insertion into a bilayer membrane but below that required for hemolysis of red blood cells.

In an aspect, the efflux pump is a bacterial drug efflux pump.

In an aspect, the efflux pump is a member of the SMR family.

In an aspect, the peptide comprises 5, 6, 7, 8, 9, 10, 11, 12, 13, 14, 15, 16, 17, 18, 19, 20, 21, 22, 23, 24, 25, 26, 27, 28, 29, or 30 amino acid residues, such as 13 amino acid residues.

In accordance with an aspect, there is provided a composition comprising the peptide described herein.

In an aspect, the composition further comprises an antibiotic, disinfectant, or chemotherapeutic.

In an aspect, the peptide and the antibiotic, disinfectant, or chemotherapeutic act synergistically.

In accordance with an aspect, there is provided a combination comprising an antibiotic and the peptide described herein.

In an aspect, the combination synergistically treats an infection.

In an aspect, the antibiotic and peptide are in the same composition.

In an aspect, the antibiotic and the peptide are in separate compositions.

In accordance with an aspect, there is provided a method of reducing multidrug resistance, the method comprising administering the peptide described herein to a subject in need thereof.

In accordance with an aspect, there is provided a method of treating an infection, the method comprising administering the peptide described herein to a subject in need thereof.

In an aspect, the method further comprises administering an antibiotic to the subject.

In accordance with an aspect, the is provided a method of improving the activity of a disinfectant, the method comprising applying the peptide described herein to a surface simultaneously or sequentially with the disinfectant.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### DESCRIPTION OF THE FIGURES

The present invention will be further understood from the following description with reference to the Figures, in which:

FIG. 1. (A) Structure of EmrE. Anti-parallel EmrE dimer (the SMR from E. coli) bound in the substrate cavity to tetraphenylphosphonium (TPP+) at 3.8 Å resolution. Adapted from, [Chen et al., 2007, Proc. Natl. Acad. Sci. U.S.A 104, 18999-19004]. (B) Molecular model of a prototypical SMR dimer with the TM4-TM4 ‘Gly heptad’ interfacial region circled. Adapted from [Rath and Deber, 2008, Proteins 70, 786-793].

FIG. 2. Crystal structure of AcrB (2DHH). (A) Formation of trimer with interactions between TM1 and TM8 at each interface. Blue and orange residues are introduced into the original figure to highlight the TM1/TM8 interaction. (B) Detail from (A), showing TM1/TM8 interaction, and indicating mutants that were shown to disrupt the AcrB trimer. (C) Stick model of TM1 (blue) and its interactions with TM8 (magenta). The full sequence of TM1 is ⁹PIFAWVIAIIIMLAGGLAILKL³⁰; TM8 is ⁸⁷¹NQAPSLYAISLIVVFLCLAALYESWSI⁸⁹⁷; projected TM8 interfacial residues are depicted in orange. Adapted from [Yu et al., 2011, PLoS ONE, 6(12), 1-8 and Ye et al., 2014, Biochemistry 53, 3738-3746].

FIG. 3. Disruption of protein-protein interactions in SMR pumps. A peptide (upper left) with the ‘Gly-heptad’ interaction sequence within TM4 competes out the native SMR TM4-TM4 helix-helix interaction (FIG. 1B) by inserting into the membrane in an antiparallel manner, and interacting at the target locus with TM4 of a given monomer—thereby preventing dimerization and consequently inhibiting efflux of substrate. The molecule shown is the toxicant ethidium bromide.

FIG. 4. (A) Proof-of-concept: Inhibition of Hsmr activity by an SMR TM4-mimic peptide. Hsmr-mediated efflux of the fluorescent toxicant ethidium bromide (EtBr) from E. coli cells upon treatment with a peptide derived from Hsmr TM4(85-105) [sequence shown]. Curves represent the change in EtBr fluorescence upon exiting the bacteria. Ac-A=acetyl-Ala. The black line indicates efflux by Hsmr in the absence of peptide; red line=efflux after addition of 1 μM TM4 peptide. The % efflux is compared in the black and red bars at the right. (B) Bioactivity profiles of designed SMR drug efflux inhibitor peptides. Four assays—EtBr efflux activity (Efflux), EtBr resensitization (ER), antimicrobial activity (AA), and hemolytic activity in human red blood cells (RBC) are depicted for linear peptides TM4(85-105) (left panel), and TM4(88-100) (centre); and stapled (S) peptide S-TM4(88-100)-N95 (right). Values for ER, AA and RBCs are given as the minimal inhibitory (MIC) or hemolytic concentration (MHC) (right axis).

FIG. 5. (A) Ethidium bromide efflux from E. coli bacteria expressing Hsmr. Peptides were added to ethidium bromide (EtBr) treated cells and incubated for 1 hr. The fluorescence of EtBr drops as the toxin is effluxed from the cells. ‘No peptide’ (maximum efflux control) is depicted in black. The core sequences of the peptides studied are based on the consensus sequence in Table 1. LII, ⁸⁸IIGIALIIAGVVV¹⁰⁰; LIN, IIGIALINAGVVV; LIN2′, IIGIALINAGVNV; and LIN2 (Nt), NIGIALINAGVVV, tagged as shown in FIG. 4A. Residues in red emphasize retention of the common SMR TM4-TM4 interface in pathogenic bacteria. S-TM(88-100)-95 is the stapled peptide shown in FIG. 4B. (B) EtBr efflux from E. coli containing plasmid-borne SMR from P. aeruginosa, (Psmr), as mediated by peptide LIN (full sequence=Ac-A(Sar)₃-⁸⁸IIGIALINAGVVV¹⁰⁰-KKK-NH₂). This experiment was performed as described in FIG. 4. The black line is Psmr-mediated EtBr efflux; the orange line shows the inhibition of efflux upon addition of 8 μM peptide.

FIG. 6. Introduction of a peptide ‘staple’ into an SMR TM4 peptide. (A) The “Grubbs metathesis” reaction. During solid phase synthesis, two non-natural amino acids (pentenyl-Ala derivatives) replace the two wild type Ala residues in a 1-5 disposition. The ‘metathesis’ reaction uses a ruthenium catalyst, which combines two olefinic sites into one. Mass spec and HPLC profiles are shown as insets. (B) Metabolic stability of unstapled vs. stapled peptide in human blood plasma and in bovine liver homogenates. Fluorescently-labelled 10 μM peptides [TAMRA-TM4(88-100) and TAMRA-S-TM4(88-100)-N95] (S=stapled) were incubated in the respective media for the indicated time periods. Cleavage products are identified by mass spectrometry. Decay of the original compound (%) was determined by peak integration. Dashed lines (top of each panel)=stapled peptide; solid lines=unstapled.

FIG. 7. Resensitization of bacteria to growth inhibition by the disinfectant benzalkonium chloride (BZK). In this experiment, a TM4 peptide consisting of the TM4 mid-sequence of P. aeruginosa was added to E. coli (BL21 strain) that contained a plasmid for overexpression of the SMR from P. aeruginosa (termed Psmr)—a situation that mimics ‘superbugs.’ The graph depicts the conditions where the bacteria were treated with either a sublethal concentration of BZK or a non-toxic dose of TM4 peptide, neither of which individually significantly inhibited bacterial growth. However, when the BZK and TM4 peptide were both present at the same concentrations, the bacterial growth was reduced to <1% [i.e., inhibited to >99% (arrow)]. As a control, the same experiment was performed with a ‘scrambled’ version of TM4 peptide (termed SCR control), having identical amino acid composition but lacking the GG7 motif for binding to Psmr and disruption of its native dimer. As shown in the graph, neither the mixture of BZK+SCR peptide nor the SCR peptide alone inhibited bacterial growth, thereby demonstrating the specificity of the inhibition process. TM4 peptide=Ac-Ala-(Sar)₃-LLGIGLIIAGVLV-KKK—NH₂ (see also Table 1). SCR control peptide=Ac-Ala-(Sar)₃-LLVLAGIGIIGLV-KKK—NH₂.

### DETAILED DESCRIPTION

Described herein are inhibitors of multimeric membrane-embedded proteins. The SMR efflux pump is given as an example, however, the methods described herein are applicable against a wide range of membrane-embedded proteins. For example, the strategies described herein could be extended to the resistance-nodulation-cell division (RND) pump—a second major pump that is a complex of the proteins AcrA-AcrB-TolC responsible for removing toxic substrates from the cell and periplasmic space, within which AcrB is a membrane-embedded obligate trimer; AcrB contains a heterogeneous TM-TM interaction site susceptible to disruption [Ye, C., et al., 2014, Biochemistry 53, 3738-3746].

The SMR efflux pump inhibitors described herein are active against a range of bacteria as many pathogenic bacteria have the same SMR TM4-TM4 sequence motif. In aspects, the inhibitors are provided in macrocyclic (‘stapled’) forms to promote their in vivo metabolic stability. In aspects, the inhibitors described herein resensitize bacteria to conventional antibiotics and in aspects act synergistically with antibiotics.

The strategy described herein of preventing protein-protein interactions using membrane-penetrating peptides to inhibit bacterial drug efflux pumps constitutes a novel approach to reduce MDR to a level that can be managed by current antimicrobials.

Over the last two decades, a notable increase in the use of ‘biologics’ has been observed in the clinic, with more than 60 FDA-approved peptide medicines now available, and many advancing to clinical trials. Peptides provide higher specificity for their targets than small molecules, and as such are expected to display lower general toxicity. The peptides described herein, in aspects, can be combined with existing antibiotics to rescue their function; can supplement treatments with inhaled antibiotics in cystic fibrosis therapy; and/or can be used to sterilize surfaces in operating rooms and food-processing facilities. The fact that the protein-protein interaction site is conserved among both chromosomal and plasmid-borne SMRs of common pathogens, allows the development of broad-spectrum inhibitors that are active against a wide range of bacteria. As many disease states are directly associated with the anomalous folding/subunit interactions of membrane proteins, the present approach is not limited to microbes and can find applications to an immense range of potential TM-TM helix-helix targets of medical importance.

### Definitions

For purposes herein, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

As used herein, “treatment” or “therapy” is an approach for obtaining beneficial or desired clinical results. For the purposes described herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” and “therapy” can also mean prolonging survival as compared to expected survival if not receiving treatment or therapy. Thus, “treatment” or “therapy” is an intervention performed with the intention of altering the pathology of a disorder. Specifically, the treatment or therapy may directly prevent, slow down or otherwise decrease the pathology of a disease or disorder such as an infection, or may render the cells more susceptible to treatment or therapy by other therapeutic agents.

The terms “therapeutically effective amount”, “effective amount” or “sufficient amount” mean a quantity sufficient, when administered to a subject, including a mammal, for example a human, to achieve a desired result, for example an amount effective to treat an infection. Effective amounts of the compounds described herein may vary according to factors such as the disease state, age, sex, and weight of the subject. Dosage or treatment regimes may be adjusted to provide the optimum therapeutic response, as is understood by a skilled person.

Moreover, a treatment regime of a subject with a therapeutically effective amount may consist of a single administration, or alternatively comprise a series of applications. The length of the treatment period depends on a variety of factors, such as the severity of the disease, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The compounds described herein may, in aspects, be administered before, during or after treatment with conventional therapies for the disease or disorder in question, such as an infection.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

Included herein are pharmaceutically acceptable salts, solvates and prodrugs of the compounds described herein and mixtures thereof.

“Stapling,” “hydrocarbon-stapling” as used herein introduces into a peptide at least two moieties capable of undergoing reaction to promote carbon-carbon bond formation that can be contacted with a reagent to generate at least one cross-linker between the at least two moieties. Stapling provides a constraint on a secondary structure, such as an alpha helix structure. The length and geometry of the cross-linker can be optimized to improve the yield of the desired secondary structure content. The constraint provided can, for example, prevent the secondary structure to unfold and/or can reinforce the shape of the secondary structure. A secondary structure that is prevented from unfolding is, for example, more stable.

A “stapled” peptide is a peptide comprising a selected number of standard or non-standard amino acids, further comprising at least two moieties capable of undergoing reaction to promote carbon-carbon bond formation, that has been contacted with a reagent to generate at least one cross-linker between the at least two moieties, which modulates, for example, peptide stability.

The compounds, proteins, or peptides described herein (e.g., amino acids, and unstapled, partially stapled, and stapled peptides and proteins) may exist in particular geometric or stereoisomeric forms. Contemplated herein are all such compounds, including cis- and trans-isomers, R- and S-enantiomers, diastereomers, (D)- and (L)-isomers, the racemic mixtures thereof, and other mixtures thereof.

Where an isomer/enantiomer is preferred, it may, in some embodiments, be provided substantially free of the corresponding enantiomer, and may also be referred to as “optically enriched.” “Optically enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); Wilen, Tables of Resolving Agents and Optical Resolutions p. 268 (E. L. Eliel, Ed., Univ. of Notre Dame Press, Notre Dame, Ind. 1972).

A “peptide,” “protein,” “polypeptide,” or “peptidic” comprises a polymer of amino acid residues linked together by peptide (amide) bonds. The term(s), as used herein, refers to proteins, polypeptides, and peptide of any size, structure, or function. Typically, a peptide or polypeptide will be at least three amino acids long. A peptide or polypeptide may refer to an individual protein or a collection of proteins. The proteins described herein typically contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A peptide or polypeptide may also be a single molecule or may be a multi-molecular complex. A peptide or polypeptide may be just a fragment of a naturally occurring protein or peptide. A peptide or polypeptide may be naturally occurring, recombinant, or synthetic, or any combination thereof.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” (or vice versa) wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation, such as any specific sequences whether implicitly or explicitly defined herein.

In addition, all ranges given herein include the end of the ranges and also any intermediate range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Bacterial Resistance Via Drug Efflux Pumps

Drug efflux pumps are widespread among bacteria, and can be divided into five main families: the small multidrug resistance (SMR) family, the resistance-nodulation-division (RND) family, the major facilitator superfamily (MFS), the multidrug and toxic compound extrusion (MATE) family, and the ATP-binding cassette (ABC) family. A majority of sequenced prokaryotes contain at least one SMR homolog, for which the native function is to remove quaternary ammonium compounds that are synthesized during metabolic activities from the cytoplasm. While SMRs have not been universally tested against all antibiotics, antibiotics have been widely reported as substrates (e.g., tobramycin, tetracycline, erythromycin, and ciprofloxacin), generally in instances where the SMRs reside on transmissible genetic elements. RND proteins also export various large cationic and hydrophobic molecules, as well as antibiotics such as erythromycin, tetracycline and tobramycin. Both SMRs and RNDs extrude disinfectants (also termed “biocides”) commonly utilized in hospitals, such as benzalkonium chloride, cetylpyridinium chloride and chlorhexidine; and dyes and other toxic substances.

### Structure of the SMR Efflux Pump

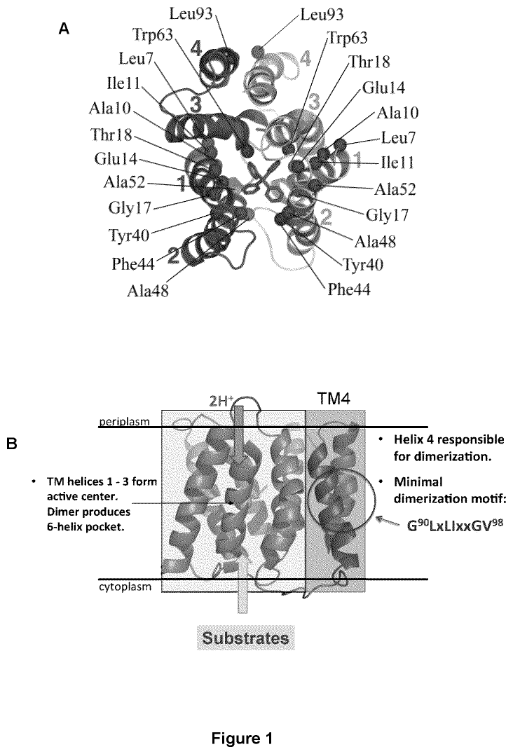

SMRs function by coupling the extrusion of one drug molecule from the cytosol to the import of two protons via the proton-motive force among which the most extensively characterized SMR is EmrE from E. coli. Despite being relatively small proteins of ˜110 residues with four TM helices, SMRs act as a ‘channel’ that can extrude molecules of the diverse sizes and shapes of antibiotics and biocides. SMR monomers must accordingly assemble into larger structures—minimally dimers—via protein-protein interactions to create a substrate pathway. Structural elucidation—cryo-EM images, and an X-ray Ca model of tetraphenylphosphonium (TPP)-bound EmrE (FIG. 1A)—have defined its architecture as an anti-parallel SMR dimer: TM's 1-3 from each EmrE monomer come together to form a hydrophobic 6-helix substrate-interaction pocket, while the two TM4 helices interact to form a dimerization arm (FIG. 1B).

### Structure of the RND Efflux Pump

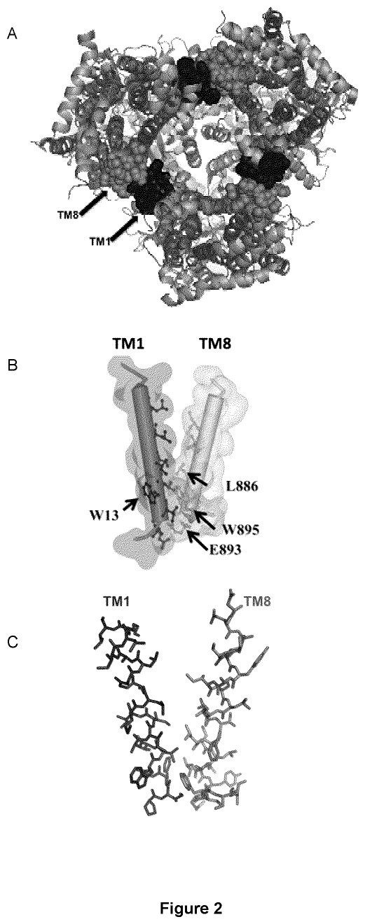

The tripartite complex AcrA/AcrB/TolC—the major RND efflux pump system within E. coli—spans the membrane and periplasmic space. It has been extensively studied since it acts as a model system for other RND superfamily pumps present in many species such as P. aeruginosa. AcrB is responsible for facilitating a peristaltic pump mechanism to remove substrates from the inner membrane or periplasmic space. AcrA docks the complex to the inner membrane while TolC spans the outer membrane. As single-component and multi-component efflux pumps, such as SMRs and RNDs, can function in series to have a synergistic effect on resistance, strategies that target both of these efflux pumps constitute an enhanced approach to significantly reducing resistance. AcrB is functional as an obligate trimer with each identical protomer cycling through three conformations powered by a proton-motive force. While extensive efforts have been made toward discovering a compound that can effectively and safely inhibit the efflux ability of AcrB, currently no such drug has been found. The most studied compound is phenylalanylarginine-β-naphthylamide (PAβN), which inhibits efflux by blocking the substrate binding pocket, has been shown to be unacceptably toxic as it disrupts membrane integrity. Thus, rather than targeting this binding pocket, described herein is an inhibitor of AcrB trimer formation (crystal structure shown in FIG. 2A). Mutagenesis studies have shown after trimer stability is decreased below a certain threshold, efflux activity is significantly reduced. While mutations within this interaction successfully reduced efflux activity, mutations that created defects in packing at TM1-TM8 interaction site (L886, E893, and W895) (FIG. 2B,C) within the membrane reduced trimer affinity and efflux activity by orders of magnitude.

Protein-protein interactions between membrane-spanning helices

Ubiquitous in protein biology, protein-protein interactions (termed “PPIs”) are the non-covalent side chain-side chain interactions—predominantly van der Waals packing and electrostatic contacts—that stabilize protein dimers and higher order oligomeric, multisubunit complexes. Given that many membrane proteins function in complexes, strategies targeting their PPIs can directly impact their function. Where protein-protein interactions occur in membranes, they are mediated by the specific topological surfaces that membrane-spanning helices present to each other. Membrane protein folding is commonly facilitated by the classically described ‘small-(x)ₙ-small motif’. Here, two small residues (Gly, Ala, Ser, or Cys) separated in the sequence by one full turn of the TM helix (n=3), are positioned on the same ‘face’ of the helix, creating a concave-like surface that promotes association with a neighboring helix through ‘knobs-into-holes’ van der Waals packing; a larger residue such as Val will obviate this packing advantage. The ‘small-small motif’ also extends to the ‘heptad’ form (n=6), where two small residues form a packing face along two turns of the helix (the ‘holes’), often accompanied by a mid-motif large residue that provides a ‘knob’ for the associating helix. In this context, one can appreciate the requirement for high specificity of PPIs in membranes, both for folding of monomers and for homogeneous or heterogeneous interactions between subunits. Lacking specificity, membrane proteins diffusing laterally through an already-crowded bilayer would adhere to each other non-specifically, eventually forming unwieldy non-functional aggregates.

### Directing Peptides to Membrane-Embedded Drug Targets

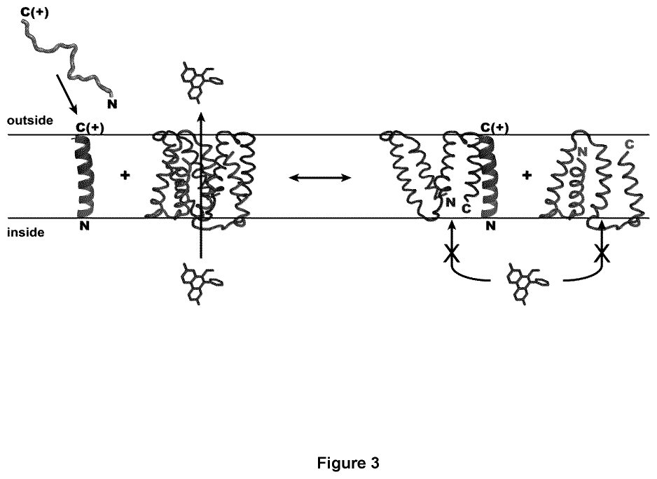

Prominent examples of functional PPIs are those central to bacterial multidrug resistance (MDR) where dimers or trimers of membrane-embedded pumps are required for their function. Thus, an approach to the development of membrane-penetrating peptides with a high potential to disrupt helix-helix association is the use of native TM sequences as templates (FIG. 3). In this regard, a single TM peptide can compete for native, intramolecular helix-helix interactions within the full-length protein, resulting in a loss of overall quarternary structure and abrogation of function. However, successful development of a membrane-insertable peptide targeting a membrane-embedded interaction site depends upon a design where (i) the peptide is of suitable hydrophobicity and positive-charge to partition into bacterial but not host membranes; (ii) the peptide can penetrate (‘burrow’) into the lipid interior of the membrane toward its target locus where it adopts a conformation complementary to the selected target; and (iii) the peptide is convenient to prepare. Described herein are methods of designing peptides to meet these goals and specific examples of peptides that achieve these goals.

### (i) Hydrophobicity Thresholds

Earlier, our laboratory defined a ‘first hydrophobicity threshold’ as the minimal segmental hydrophobicity—averaged over the amino acid sequence from hydrophobicity scale—that is required for peptide insertion from water into SDS micelles or anionic (bacteria-like) bilayer membranes [Liu et al., 1996, Biopolymers 39, 465-470]; using standard hydropathy scales [Deber et al., 2001, Protein Sci. 10, 212-219], this threshold equates to average hydropathy just above that of a poly-Ala segment. We then established [Glukhov et al., 2008, Biopolymers 89, 360-371] that hydrophobic peptides do not disrupt (hemolyze) human red blood cell membranes (RBCs)—which are neutral (zwitterionic) rather than anionic like bacterial membranes—until the average peptide hydrophobicity exceeds a ‘second hydrophobicity threshold’, which will occur for segments overly rich in Leu, Val, or Ile residues. Knowledge of these two thresholds significantly narrows peptide design towards those that exceed the first—but not the second—threshold, thus ensuring safety for host cell membranes.

### (ii) Membrane Insertion and Orientation: Peptide Tagging.

To achieve the desired ‘burrowing’ property, and ensure the preferred anti-parallel orientation of the inhibitor peptide vis-à-vis its TM4 target, the peptide design includes an uncharged N-terminus that would allow peptide insertion into the membrane. Thus, we developed a terminal tag (either N- or C-terminal) that contains an acetylated N-terminus (Ac-Ala) linked to three sarcosine (N-methyl-Gly) residues (Sar)₃. Residues such as Sar—termed ‘peptoids’ to indicate their N-substituent—aid in the solubilization of hydrophobic TM peptides. Further, to ensure the water solubility of these hydrophobic peptides during workup, we borrowed the feature that most TM segments in membrane proteins are flanked by Lys (or Arg) residues at membrane entry/exit points, and routinely lag′ core sequences by Lys residues at the N- and/or C-termini. We earlier confirmed that Lys-tagged TM peptides with naturally-derived sequences are capable of reproducing the oligomeric states of the corresponding intact biomolecules [Melnyk et al., 2001, Biochemistry 40, 11106-11113; Tulumello and Deber, 2012, Biochim. Biophys. Acta. 1818, 1351-1358].

(iii) Peptide Synthesis and Characterization

Peptides were synthesized on a 0.1 mmol scale using standard solid-phase Fmoc chemistry [Cuff and Oullette, 2010, Molecules 15, 5282-5335; Melnyk et al., 2003, Biopolymers 71, 675-685; Merrifield, 1969, Adv. Enzymol. Relat. Areas Mol. Biol. 32, 221-296; Liu and Deber, 1997, Biochemistry 36, 5476-5482, Poulsen and Deber, 2012, Antimicrob. Agents Chemother. 56, 3911-3916]. Peptides were cleaved from solid supports with a cocktail of TFA/phenol/ultrapure water/triisopropylsilane and precipitated from ether. Mass spectrometric analysis of crude chromatograms confirmed desired products. Purification of the peptides was achieved on C4 preparative HPLC columns in linear water/organic gradients. An automated peptide synthesis facility performs peptide syntheses routinely.

First Generation Inhibitors: Inhibition of Drug Efflux from Bacteria;

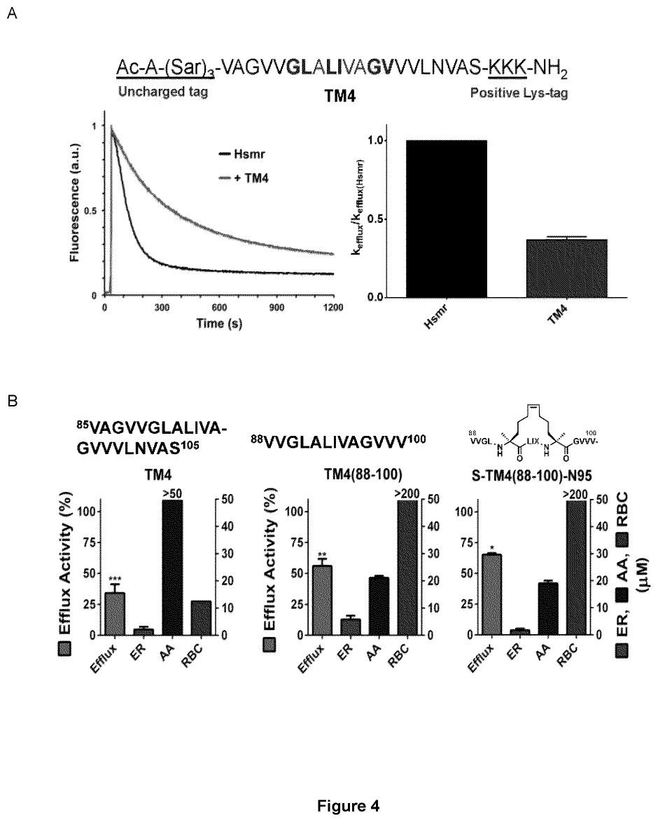

A Gly-based heptad repeat motif in the fourth TM helix of Hsmr (a lab model SMR from Halobacterium salinarum [Poulsen and Deber, 2009, J. Biol. Chem. 284, 9870-9875]) required for dimerization and essential for function (centered on TM4 residues ⁹⁰GxxLIxxG⁹⁷V⁹⁸) was identified [Poulsen et al., 2009, J. Bacteriology 193, 5929-5935]. Using the framework outlined above, proof-of-principle of the SMR assembly-disruption concept was shown, using a full-length Hsmr peptide [TM4(85-105): Ac-A(Sar)₃VAGVVGLALIVAGVVVLNVASKKK-NH₂] to specifically inhibit Hsmr-mediated efflux from bacteria in vivo of ethidium bromide (EtBr)—a toxic molecule for which fluorescence changes can be monitored as it is extruded from inside to outside of the cell (FIG. 4A) [Poulsen and Deber, 2012, Antimicrob. Agents Chemother. 56, 3911-3916]. As controls for specificity, we showed that the all-D isomer of TM4(85-105), and sequence-scrambled versions of the peptide with identical composition to wild type where the heptad motif is disrupted, did not inhibit EtBr efflux. To evolve this approach, we then showed that peptides shortened from the 21-residue Hsmr TM4(85-105) to a 13-residue core sequence Hsmr TM4(88-100) (the sequence centered on the ‘Gly heptad’ helix-helix interaction residues) [Bellman-Sickert et al., 2015, J. Biol. Chem. 290, 1752-1759]maintain SMR inhibitory function at levels comparable to the full-length TM4 peptide. In this phase of the work, we extended our analysis to a panel of assays to construct a “bioactivity profile” for each peptide. These assays are as follows:

### (i) Efflux Assays

EtBr efflux represents a rapid and validated means by which to screen for SMR-mediated resistance activity [Poulsen and Deber, 2012, Antimicrob. Agents Chemother. 56, 3911-3916]. Here, E. coli cells are incubated with the peptide, EtBr and carbonyl cyanide 3-chlorophenylhydrazone (CCCP), a widely-used ionophore that acts as a disruptor of the proton-motive force. Upon its removal, EtBr efflux is monitored by fluorescence decay.

(ii) Resensitization assays

Resensitization to EtBr and other toxic SMR substrates are evaluated by incubating the bacteria with a sublethal concentration of the toxicant and twofold dilutions of peptide at 37° C. for 20 hr, after which bacterial growth (or lack thereof) is evaluated [Bellman-Sickert et al., 2015, J. Biol. Chem. 290, 1752-1759]. If the peptide inhibits efflux ata given dilution, bacteria will be rendered unable to efflux the EtBr.

(iii) Bactericidal Assays

Noting that designed inhibitors may act as antibiotics—raising the possibility of their evoking non-specific membrane disruption—the minimum inhibitory concentration (MIC) of each new peptide is assayed [Yin et al., 2012, J. Biol. Chem. 287, 7738-7745] to determine its antibiotic activity, as distinct from resensitization to toxicants.

### (iv) Hemolysis Assays in Human Red Blood Cells (RBCs)

As a necessary measure for any projected clinical applications of the designed inhibitors, hemolytic activity of peptides is assayed in human RBCs to confirm mammalian cell tolerance [Yin et al., 2012, J. Biol. Chem. 287, 7738-7745].

Bioactivity profiles are shown for an initial set of peptides in FIG. 4B. While the full-length TM4 (TM85-105) is partially hemolytic to RBCs (MHC=˜10 μM) (left panel), the shortened version TM4(88-100) (center panel) exhibits a highly favourable profile with significant efflux, excellent resensitization, and total absence of hemolysis (MHC>200 μM).

### Second-Generation Inhibitors

The design and synthesis of the next generation of prospective drug efflux inhibitors is now described, with focus on preparing peptides that are the most potent, have the greatest metabolic stability, and ultimately those with the broadest range of toxicant efflux inhibition activity against MDR bacteria. The following specific approaches toward these goals are described:

### Design of Broad-Spectrum SMR Peptide Inhibitors

Noting that the SMR sequence homology for the TM4-TM4 motif is striking across many common pathogens, including S. aureus, P. aeruginosa, and M. tuberculosis, we have evolved our original designs to synthesize inhibitors against these bacteria, as well as new broad-spectrum inhibitors of the SMR efflux pump. Peptide bioactivity will be optimized through iterations of peptide length, sequence hydrophobicity, and N- and C-terminal substituents. Bioactivity profiles will be generated for each new peptide, assessing toxicant efflux, resensitization and antibiotic MICs, and safety toward human red blood cells.

### Engineering Peptide Metabolic Stability

To create more proteolysis-resistant analogs, the best peptides emerging from this screen will be chemically ‘stapled’, and the resulting macrocyclic peptides compared with linear counterparts for relative bioactivity and metabolic stability in vivo.

### Targeting the Trimerization Site in AcrB

As mutagenesis studies have identified an interaction site between TM helices 1 and 8 in AcrB as a PPI target, we will create synthetic peptides designed to effectively mimic both of the hetero-oligomeric TM1/TM8 interaction faces and perform parallel experiments to the SMR studies.

### Resensitization of Pathogenic Bacteria to Antibiotics

To determine whether the designed peptides can restore the susceptibility of bacteria to conventional antibiotics, a range of bacteria will be treated with antibiotics in the presence of inhibitor peptides, and the resulting MICs evaluated. Resensitization by SMR and AcrB inhibitors will be tested separately, and in tandem.

### Peptides

Thus, described herein are peptides that are capable of disrupting assembly of multimeric membrane-embedded proteins. The peptides described herein are generally from about 5 to about 30 peptides in length, such as from about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, or about 29 to about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 peptides in length.

Membrane-embedded proteins typically contain helical segments that span some or all of the membrane. Some membrane-embedded proteins have single-spanning helical segments and others have multi-spanning helical segments. The peptides described herein are rationally designed to be complementary to a motif within a helical transmembrane segment of a protein so as to competitively inhibit a helix-helix interaction. This can inhibit proper multimerization of proteins having one or more interacting helical segments. In this way, the activity of the protein is reduced. In aspects, the peptide inhibits multimerization of dimers, trimers, tetramers, pentamers, hexamers, and so on. The multimers may be homomultimers or heteromultimers.

In aspects, the sequence of the peptide is 100% complementary to its target. However, in other aspects, the sequence is modified so as to have broader spectrum activity against related transmembrane proteins in other species or within the same species. In such a case, the sequence of the peptide may not be 100% complementary to its target and may, rather, have at least about 50%, about 60%, about 65%, about 70%, about 75%, about 80%, about 85%, about 90%, about 95%, about 96%, about 97%, about 98%, or about 99% sequence identity to its target. In such aspects, the sequence of the peptide may be designed to be complementary to a consensus sequence and/or may contain specific residues that are complementary to residues that are conserved across species or within a given family of proteins in the same species.

In typical aspects, the peptide is helical when inserted into a membrane and may contain one more tags that facilitate membrane insertion. In aspects, the peptide contains a membrane-insertion tag and/or a solubility-increasing tag. The solubility-increasing tag is typically positively charged and increases the solubility of the peptide. The solubility-increasing tag typically comprises one or more positively charged amino acid residues, such as from about 1 to about 10 amino acid residues, such as from about 1, about 2, about 3, about 4, about 5, about 6, about 7, about 8, or about 9 to about 2, about 3, about 4, about 5, about 6, about 7, about 8, about 9, or about 10 amino acid residues. In aspects, the positively charged amino acid residues are selected from lysine, histidine, and/or arginine residues, or combinations thereof. Typically, the positively charged amino acid residues are lysine residues. The solubility-increasing tag may be located at the C- and/or N-terminus of the peptide, but is typically at the N-terminus.

The membrane-insertion tag is typically uncharged and typically comprises one or more peptoid residues, such as from about 1, about 2, about 3, about 4, about 5, about 6, about 7, about 8, or about 9 to about 2, about 3, about 4, about 5, about 6, about 7, about 8, about 9, or about 10 peptoid residues. Typically, the peptoid residues are selected from NVal (N-isopropylglycine), NLeu (N-isobutylglycine), NAla (N-methylglycine; sarcosine), Nile (sec-butylamine), NMet (2-(methylthio)ethylamine), NPhe (Benzylamine), or combinations thereof. Typically, the peptoid residues are sarcosine residues. The membrane-insertion tag may be located at the C- and/or N-terminus of the peptide, but is typically at the C-terminus.

When the membrane-insertion tag is at the N-terminus, it typically comprises an N-terminal amino group blocking moiety, which is typically N-acetylated. In typical aspects, the N-terminal amino group blocking moiety comprises an N-acetyl-Ala residue. Thus, a typical N-terminal membrane-insertion tag comprises the sequence N-acetyl-Ala-Sar-Sar-Sar-.

When the membrane-insertion tag is at the C-terminus, it typically comprises a C-terminal carboxylate group blocking moiety, which is typically methyl esterified. In typical aspects, the C-terminal carboxylate group blocking moiety comprise a Sar-methyl ester residue. Thus, a typical C-terminal membrane-insertion tag comprises the sequence -Sar-Sar-Sar-Sar-methyl ester.

In typical aspects, when the peptide is targeted to bacterial cell proteins, the peptide comprises a hydropathy value that permits insertion into bacterial membranes but not eukaryotic cell membranes. For example, according to the Liu-Deber (LD) hydropathy scale, the numbers have been scaled between −5.0 and +5.0, assigning a hydropathy value to each residue; +5 is high hydropathy. If the LD value of each residue in the peptide is added and divided by the number of residues, the LD average (or segmental) hydropathy is provided. On the LD scale, a value of approximately +0.4 is sufficient to predict that a peptide sequence will insert spontaneously from water into a membrane environment. When a value above approximately +1.8 is reached, the peptide becomes less soluble in water, and will partition into available membranes regardless of their charge or the net charge on the peptide. When this occurs, hemolysis of eukaryotic membranes becomes apparent. Thus, in aspects, the peptides described herein have a hydropathy value between about +0.4 and about +1.8, as measured on the LD scale. In aspects, the hydropathy value is between about +0.4, +0.5, +0.6, +0.7, +0.8, +0.9, +1.0, +1.1, +1.2, +1.3, +1.4, +1.5, +1.6, or +1.7 and about +0.5, +0.6, +0.7, +0.8, +0.9, +1.0, +1.1, +1.2, +1.3, +1.4, +1.5, +1.6, +1.7, and +1.8.

In typical aspects, the peptide is stapled or made to be macrocyclic by any known method. “Peptide stapling” is a term coined for a synthetic methodology used to covalently join two olefin-containing side chains present in a polypeptide chain using an olefin metathesis reaction (J. Org. Chem. (2001) 66(16); Blackwell et al., Angew. Chem. Int. Ed. (1994) 37:3281). Stapling of a peptide using a hydrocarbon cross-linker created from an olefin metathesis reaction has been shown to help maintain a peptide's native conformation, particularly under physiological conditions.

In certain aspects, the peptide is designed specifically to inhibit the activity of a drug efflux pump, such as those found in cancer cells or bacterial cells. In aspects, the peptide inhibits one or more members of the small multidrug resistance (SMR) family, the resistance-nodulation-division (RND) family, the major facilitator superfamily (MFS), the multidrug and toxic compound extrusion (MATE) family, or the ATP-binding cassette (ABC) family, or combinations thereof.

For example, when the peptide inhibits a member of the SMR family, the peptide may comprise the sequence:

X₁-X₂-(G or S)-X₃-X₄-L-(I or M)-X₅-X₆-G-(V or I)-X₇X₈

wherein each of X₁-X₈ is independently any amino acid and is independently present or absent; wherein the peptide has fewer than 30 amino acid residues; and wherein the peptide binds to and inhibits an efflux pump, with the proviso that the peptide does not comprise VVGLALIVAGVVV or VVGLALINAGVVV. In typical aspects, each of X₁-X₈ is present.

In aspects, X₁ is V, F, I, L, or C; X₂ is V, I, or L; X₃ is L, M, or I; X₄ is A, G, I, M, V, or L; X₆ is V, I, C, or G; X₆ is A, S, V, F, C, or T; X₇ is V, L, or I; and X₈ is V, T, L, or I. Optionally, up to three of X₁-X₈ are independently replaced with a residue selected from N and Q so as to reduce the overall hydrophobicity of the peptide.

In aspects, the peptide comprises one of the following sequences:

FVGMGLIVSGVVV;

VVGIGLIVVGVVT;

IISIILIIFGVVL;

LLGIGLIIAGVLV;

CIGLALMIAGIVI;

IIGMMLICAGVLV;

IVSIVLIIVGVVL;

IIGMLLIICGVIV;

IIGMMLICTGVLV; or

IIGIGLIIAGVVV, each of which may further comprise a solubility-increasing tag and/or a membrane-insertion tag as described above.

In other typical aspects, the peptide comprises the sequence IIGIGLIIAGVVV, or a fragment or variant thereof having at least 50%, 60%, 70%, 75%, 80%, 85%, 90%, 95%, 96%, 97%, 98%, or 99% identity to IIGIGLIIAGVVV. Typically, the peptide has fewer than 30 amino acid residues and binds to and inhibits a drug efflux pump, with the proviso that the peptide does not comprise VVGLALIVAGVVV or VVGLALINAGVVV.

In other typical aspects, the peptide comprises about 5 to about 30 amino acid residues and binds to and inhibits interactions between two or more transmembrane regions of a drug efflux pump, thereby inhibiting the drug efflux by the pump, with the proviso that the peptide does not comprise VVGLALIVAGVVV or VVGLALINAGVVV.

In other typical aspects, the peptide competitively inhibits multimerization of a membrane-embedded protein, wherein the peptide is complementary to a motif within a helical transmembrane segment of the protein and thereby competitively inhibits a helix-helix interaction between monomers to inhibit multimerization of the protein.

These peptides may comprise one or more tags, as described above, and may be stapled.

### Compositions

The peptides described herein, in aspects, are formulated into compositions. The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, 20th ed., Mack Publishing Company, Easton, Pa., USA, 2000). On this basis, the compositions may include, albeit not exclusively, the peptides in association with one or more pharmaceutically acceptable vehicles or diluents, and may be contained in buffered solutions with a suitable pH that are iso-osmotic with physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of the subject. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The pharmaceutical composition may be supplied, for example, but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the active agent, together with a suitable amount of carrier so as to provide the form for direct administration to the patient.

### Methods of Use

The peptides described herein can be rationally designed to target any multimeric membrane-embedded protein and thus find use in treating any disorder in which inhibition of such a protein would be useful. Examples include bacterial infections or cancer, wherein the peptides may be designed to inhibit drug efflux pumps. To this regard, it is contemplated that the peptides described herein may be used in combination with conventional treatments for infection or cancer, such as antibiotics or chemotherapy, resulting in an additive or synergistic treatment modality.

The peptides described herein can, in aspects, be administered for example, by parenteral, intravenous, subcutaneous, intradermal, intramuscular, intracranial, intraorbital, ophthalmic, intraventricular, intracapsular, intraspinal, intracisternal, intraperitoneal, intranasal, intrarectal, aerosol or oral administration. Typically, the compositions described herein are administered subcutaneously, intramuscularly, intradermally, or by inhalation.

The peptides may, in aspects, be administered in combination, concurrently or sequentially, with conventional treatments for infection or cancer, including antibiotics, anti-inflammatory agents, chemotherapy, hormone therapy, biotherapy, and radiation therapy, for example. The peptides may be formulated together with such conventional treatments when appropriate. For example, the peptides may be administered prior to conventional treatments so that the bacteria or cancer cells are rendered more susceptible to the conventional treatments.

The peptides may be used in any suitable amount, but are typically provided in doses comprising from about 0.001 μM to about 1000 μM peptide, such as from about 0.001 μM, about 0.01 μM, about 0.1 μM, about 1 μM, about 10 μM, or about 100 μM to about 0.01 μM, about 0.1 μM, about 1 μM, about 10 μM, about 100 μM, or about 1000 μM agonist. Alternatively, the peptides may be administered in doses such as from about 0.001 mg/kg to about 1000 mg/kg, such as from about 0.001 mg/kg, about 0.01 mg/kg, about 0.1 mg/kg, about 1 mg/kg, about 10 mg/kg, or about 100 mg/kg to about 0.01 mg/kg, about 0.1 mg/kg, about 1 mg/kg, about 10 mg/kg, about 100 mg/kg, or about 1000 mg/kg.

Additionally, treatment with the peptides described herein may occur once or may be repeated several times. For example, treatment may occur daily, weekly, monthly, yearly, or a combination thereof, depending upon the disease state. For example, a subject may be administered several doses on an hourly, daily, or weekly basis in order to treat an active infection. Once the infection slows or goes into remission, follow-up maintenance doses may be provided, for example, on a monthly basis, every three months, every six months, or on a yearly basis, or simply as needed at the sign of any return of infection.

It will be understood that the peptides, while useful for resensitizing bacteria to antibiotics, may themselves have an antibiotic effect by virtue of their ability to enter the cell membrane. Therefore, methods of treatment of an infection comprising administering the peptides described herein are also contemplated.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1—Design of Peptide Inhibitors of SMR-Mediated Drug Efflux from Pathogenic Bacteria

### Conservation of the Dimerization Motif in Bacterial SMRs.

The great potential of the TM4-based peptide inhibitors to target SMR dimerization sites became apparent when we examined the conservation of the essential residues of the SMR heptad repeat motif. In comparative genomics searches we performed of bacterial SMRs, we observed that the TM4-residue homology is striking across a spectrum of organisms that spans species such as E. coli, P. aeruginosa, S. aureus, M. tuberculosis, K. pneumonia, E. faecium, and A. baumannii. (Table 1). Remarkably, the SMRs of all of the CDC's “ten top threat” bacteria contain a virtually identical ‘face’ of TM4-TM4 interaction motif [viz., ⁹⁰GLALIVAGV⁹⁸ in Hsmr; 100% conserved residues underlined], suggesting a common dimerization mechanism for the SMRs of all of these bacteria. Parallel searches revealed no comparable mammalian protein sequences, thereby reducing the likelihood of off-target effects.

### Designs Tailored to SMRs of Individual Bacteria.

Now described are synthesized peptides of prototypical sequence [Tag₁-(TM4)88-100))-Tag₂], where the 88-100 sequence consists of residues corresponding to individual TM4 sequences of selected bacteria, including, for example, those of P. aeruginosa and S. aureus; Tag₁ and Tag₂ as in FIG. 4A. The ‘Gly heptad’ motif will be preserved while exploring the variants that arise in the surrounding residues. Peptoid residues will be utilized to ensure that the peptide N-terminus is charge-neutral. The number and type of peptoid ‘tag’ residues [e.g., NVal (with an N-isopropyl group) or NLeu (with an N-isobutyl group)] will be varied to optimize overall peptide penetration and hydrophobicity. Lys content will also be varied (2-6 residues). Lead inhibitors will be identified through bioactivity profiles as described above.

TABLE 1
Multiple sequence alignment of transmembrane helix 4
in Hsmr to TM4 sequences in SMRs of pathogenic bacteria.

| | | | % sequence identity | |
|---|---|---|---|---|
| Top antibiotic | Gram | | to Hsmr^{f} | |
| resistant threats | positive or | Transmembrane helix 4 | Full-length | TM4 |
| in the US, 2013.^{a} | negative^{b} | aligned sequences^{c} | protein | 85-105 |
| H. salinarum | − | VAGVVGLALIVAGVVVLNVAS^{d} | 100 | 100 |
| K. pneumonia (CRE) | − | AWGFVGMGLIVSGVVVLNLLS | 47 | 79 |
| M. tuberculosis | + | VMKVVGIGLIVVGVVTLNLAG | 44 | 71 |
| E. faecium (VRE) | + | LISIISIILIIFGVVLLNTFG | 40 | 70 |
| P. aeruginosa | − | PAALLGIGLIIAGVLVIQLFS | 49 | 64 |
| A. baumannii | − | LAACIGLALMIAGIVIINVFS | 45 | 60 |
| E. coli (CRE) | − | LPAIIGMMLICAGVLVINLLS | 44 | 53 |
| S. aureus (MRSA) | + | LITIVSIVLIIVGVVLLNIFG | 40 | 50 |
| S. enterica | − | MPAIIGMLLIICGVIVINLFS | 38 | 47 |
| S. flexneri | − | LPAIIGMMLICTGVLVINLLS | 42 | 47 |
| Consensus sequence^{e} | | LAAIIGIGLIIAGVVVLNLFS | | |

^{a}H. salinarum is compared to antibiotic resistant bacteria species identified as urgent threats by the CDC. CRE = carbapenem-resistant Enterobacteriaceae. VRE = vancomycin resistant Enterococci. MRSA = Methicillin-resistant Staph. aureus.
^{b}Bacteria and archaea (H. salinarum) responses to gram staining.
^{c}Multiple sequence alignment of transmembrane helix 4 from SMR efflux pumps. Highly conserved residues (≥60% occurrence, Jalview [Poulson and Deber, 2012, Antimicrob. Agents. Chemother. 56:3911-3916]) are highlighted in red. Alignments were performed using annotated sequences for the SMR efflux pump from the respective bacterial species. Sequences were aligned using Blastp (protein-protein BLAST).
^{d}The full-length Hsmr TM4 sequence (Val-85 to Ser-105).
^{e}Consensus sequence as determined by Jalview [Waterhouse et al., 2009, Bioinformatics, 25:1189-1191]; amino acids are those with the highest occurrence at each position within TM4 for all 10 species; residues 88-100 are underlined.
^{f}Comparison of protein sequence percent identity for full-length protein and TM helix 4 to Hsmr, illustrating the high conservation of TM4 helices vs. the SMR protein sequences as a whole.

Toward Broad-Spectrum Inhibitors: A Consensus Sequence

The striking similarities found between the TM4 sequences of SMR proteins in various bacterial species imply a universal mechanism for SMR function. This key circumstance allows us to generate a ‘consensus sequence’ (Table 1), and prepare peptides corresponding to this consensus sequence, starting with the TM4(88-100) segment for which bioactivity has been established in Hsmr. Then, in parallel with generating the individual inhibitor peptides described above, we will obtain the genes encoding SMRs from common pathogens (e.g., A. baumannii AbeS; P. aeruginosa Psmr; S. aureus Smr) (commercially available) and subclone each into the pT7-7 vector currently utilized for EmrE and Hsmr expression. Each SMR will then be expressed in E. coli (as done for H. salinarum Hsmr) and we will test the ability of both individual and consensus sequence SMR inhibitors to inhibit activity in bacterial efflux and resensitization assays.

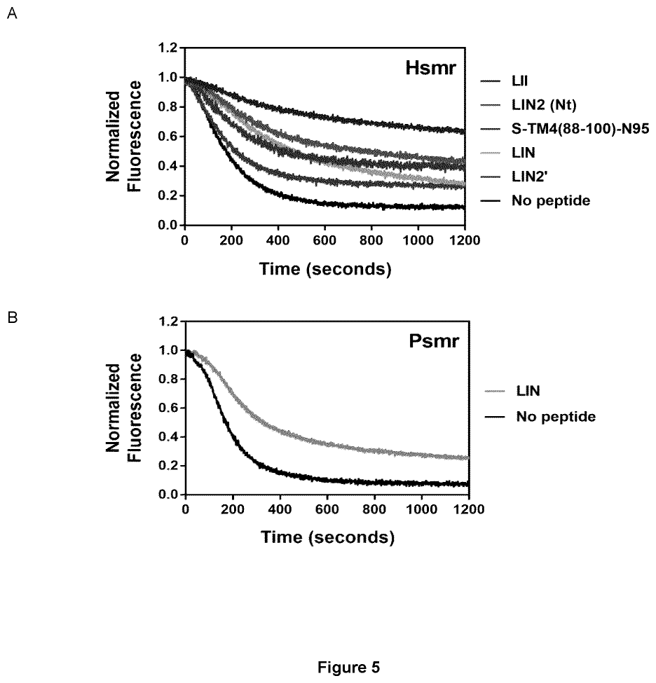

In this context, we have recently performed two pilot studies. In one study, we tested the SMR motif conservation principle by preparing peptides based on the consensus sequence of the TM4-TM4 interaction site, and tested them against E. coli bacteria containing a plasmid with Hsmr from H. salinarum. As seen in FIG. 5A, these peptides inhibit EtBr efflux up to ˜70%—a level that supports the prospect of drug efflux synergy between SMR and the RND complex. Bacterial assays we performed as controls report no significant non-specific cell death due to the peptides at the concentrations tested for efflux inhibition (not shown). In a second set of experiments, we prepared E. coli containing Psmr—the SMR from P. aeruginosa—and confirmed the peptide's ability to inhibit EtBr efflux (FIG. 5B). This extraordinary motif conservation thus allows us to drastically limit the manipulation of residues, and thereby significantly speed inhibitor development.

### Peptide ‘Stapling’ to Confer Conformational and Metabolic Stability

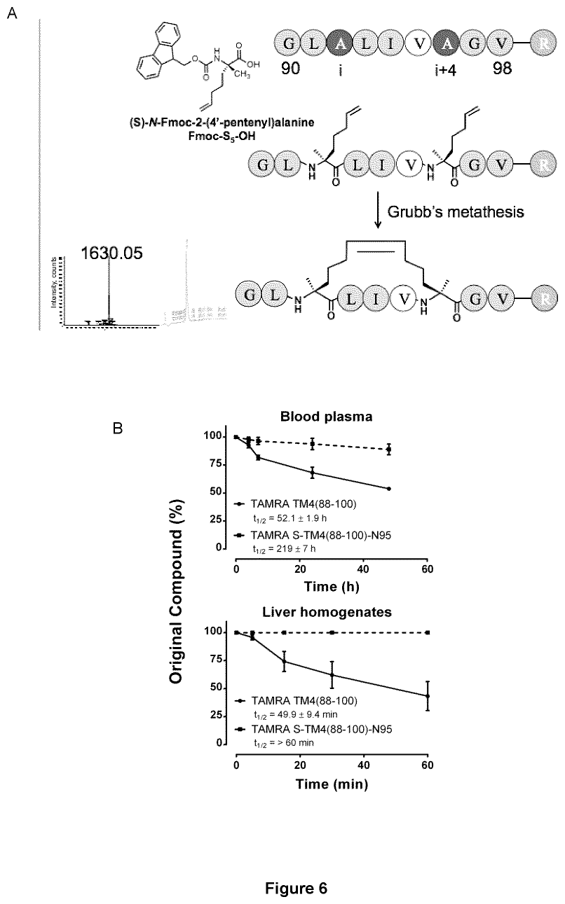

Scientists involved in peptide drug discovery are acutely aware of the susceptibility of peptides to rapid degradation and body clearance. Described herein is a route to overcoming this situation by employing a synthetic method termed ‘peptide stapling’—a strategy that has found considerable utility in pharmaceutical science [Bernal et al., 2010, Cancer Cell 18, 411-422; Aileron Therapeutics Inc. (2013). Aileron Therapeutics successfully completes first-ever stapled peptide clinical trial; Chang et al., 2013, Proc. Natl. Acad. Sci. USA 110, 3445-3454. Covalent stapling (‘macrocyclization’) enhances peptide stability and cell penetration [Melnyk et al., 2003, Biopolymers 71, 675-685]. Macrocyclization provides protection from non-specific proteolytic degradation in vivo, thereby extending peptide in vivo half-life, while ‘locking in’ the bioactive a-helical structure that specifies the peptide target. This method—termed the ‘Grubbs metathesis’ [Rosebrugh et al., 2013, J. Am. Chem. Soc. 135, 1276-1279]—is a one-step reaction using a ruthenium catalyst that allows the α-helix side chains of a peptide to be cross-linked by incorporating two α-methyl-α-alkenyl amino acids (FIG. 6A). These residues are routinely introduced during solid-phase peptide synthesis [Kim et al., 2011, Nature Protoc. 6, 761-771; Verdine and Hilinski, 2012, Methods Enzymol. 503, 3-33], after which ‘stapling’ is performed.

We have prepared prototypical examples of stapled peptides, and showed that S-TM(88-100)-N95 (S=stapled) displays an excellent bioactivity profile (FIG. 4B, right panel). The remarkable stability conferred by this strategy was then shown in a pilot study with human blood plasma and bovine liver homogenates, where we observed that the stapled analog was not degraded over lengthy periods of time [Bellmann-Sickert et al., 2015, J. Biol. Chem. 290, 1752-1759] (FIG. 6B). To our knowledge, these experiments represent the first utilization of hydrocarbon stapling to target and disrupt a membrane-embedded protein sequence. We now plan to apply the stapling technique to the best inhibitors that emerge from the SMR studies described above. It is noted that the introduction of the hydrocarbon staple increases the sequential hydrophobicity of the wild type sequence; in practice, the staple is equivalent to essentially two Leu residues. Thus, while maintaining the conserved TM4 heptad motif, we can reduce the hydropathy of other residues, viz., the change from wild type V95 to N95 in the stapled SMR peptide (FIG. 4B, right panel) eliminated hemolytic effects.

### Example 2—Peptide Inhibitors of the RND Efflux Pump

We recognize that application of the inhibitor concept to the SMR pump alone nevertheless leaves the bacteria with other options for toxicant efflux. Accordingly, in approaches parallel to those we describe for inhibiting drug efflux by SMRs, synthetic peptides will be designed corresponding to the TM helices in a second major efflux pump—the RND complex—that will disrupt the interaction interface between TM1 and TM8 that has been shown to abrogate AcrB activity when mutagenized. TM8 interacts with TM1 at the terminal end of the membrane in an antiparallel manner (FIG. 2B,C). Interestingly, the common ‘small-xxx-small’ motif occurs in TM1 near its projected interaction site with TM8 (¹²AWVIA¹⁶), along with a second site nearer the C-terminus (²²AGGLA²⁶) (full TM1 and TM8 sequences given in FIG. 2 legend). In addition, two modified ‘heptad’ motifs (where the ‘small’ residues are Cys and Ser) occur in TM8 within the TM1-TM8 interaction helix-helix interaction site (⁸⁸⁰SLIVVFLC⁸⁸⁷ and ⁸⁸⁷CxxALxxS⁸⁹⁴). Since TM8 inserts into the membrane C-terminus first, here we will add the hydrophobic tag of three Sar residues to the C-terminus of the synthetic TM8 to allow antiparallel alignment. We will prepare tagged peptides corresponding to each of the full sequences of TM1 and TM8, and as results emerge, pare them down to minimal active sequences. Stapled analogs will be then be produced.

### Example 3—Resensitization of Bacteria to Antibiotics

Our experiments-to-date have demonstrated the ability of the peptide inhibitors based on Hsmr to significantly limit ethidium bromide efflux from E. coli (FIG. 4). However, it is clear that resensitization of bacteria to conventional antibiotics, i.e., improving their MICs in the presence of peptides, would be a key determinant of the scope of the inhibitors. We thus will adapt these resensitization protocols with the goal of establishing the broadness of the inhibitory effect. Thus, bacteria will be treated with sub-lethal doses of selected conventional antibiotics, such as tobramycin and tetracycline, and then incubated with varying doses of inhibitor peptides (such as TM4(88-100), S-TM4(88-100)-N95, and newly-obtained consensus sequence peptides), and the resulting MICs (termed “ER” in FIG. 4B) will be measured. Doses will be kept below 10% of the corresponding antibiotic MICs (“AA” in FIG. 4B) to realize the inhibitor selectivity. Success here would be manifested by a decrease in resensiziation MICs vs. those of the conventional antibiotics alone. Beyond E. coli, bacteria selected from Table 1, such as P. aeruginosa, will be evaluated. We will also perform these experiments in ‘reverse’ order, i.e., pre-treating bacteria with peptide, and then administering doses of antibiotic. The lead peptides that are expected to arise from the AcrB TM1 and TM8 designs will similarly be evaluated for resensitization properties as described for the SMR inhibitors. Ultimately, we will mix examples of the two inhibitory peptide categories to create a potentially “synergistic cocktail” that in combination with traditional antibiotics should be particularly lethal to pathogenic bacteria.

### Example 4—Resensitization of Bacteria to Disinfectants (Biocides)

Hospitals, health care facilities, and the food production industry have become heavily reliant on regimens that control and prevent the spread of pathogenic bacteria. Yet, multidrug resistant ‘superbugs’ persist in these facilities because bacteria have drug resistance proteins in their membranes that pump out the antibiotics that people administer. More recently, this situation is becoming more dire because bacteria are exhibiting cross-resistance to the disinfectants routinely used to sanitize surfaces [Pal et al. (2013). Nucleic Acids Res. 42, 1814-1838].

Disinfection is typically achieved through the use of biocides—compounds that destroy or prevent the action of any harmful organism—of which benzalkonium chloride (BZK) is a widely-used example. However, while the intention is to prevent the spread of antibiotic-resistant superbugs before they infect patients, bacteria are emerging that can survive exposures to both biocides and antibiotics, viz., biocide exposure can ‘train’ superbugs to resist antibiotics—a phenomenon known as cross-resistance [Gibbons et al. (2013). Open Microbiol. J. 7, 34-52]. This means that biocide residues left on hospital surfaces after washing can promote the growth of antibiotic-resistant bacteria. For example, the osmoprotectants betaine and choline are natural quarternary ammonium compound (QAC) substrates of EmrE [Nikaido, H. (2009). Annu. Rev. Biochem. 78, 119-146], and the SMR pump NepAB of Arthrobacter nicotinovorans extrudes methylamine, the end product of nicotine catabolism by this organism [Li and Nikaido. (2009). Drugs. 69, 1555-1623].

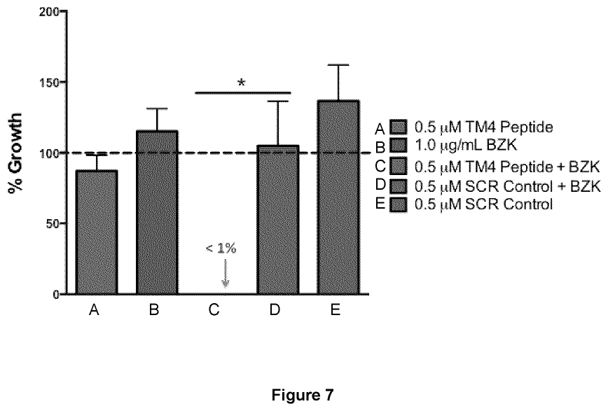

Antimicrobial peptides are exciting leads in the development of novel biocidal agents. Such peptides are currently used commercially in topical formulations, such as the lipopeptides polymyxin B and daptomycin [Kalorama Information Market Intelligence Report, “Health Care Infection Control Market” (126 reports; published on-line May, 2016)]. Further, antimicrobial peptides can enhance the performance of QAC biocides, e.g., inactivation of planktonic S. aureus was significantly enhanced when BZK and cetrimide were used in combination with the cyclic antimicrobial peptide AS-48, while BZK/AS-48 combinations decreased the concentrations of sessile and planktonic S. aureus in storage solutions [Report on Peptide Therapeutics Market (by Synthesis—SPPS, LPPS, HPPS, by Application—Cancer, Metabolic, CVS, CNS & others, by Types—Innovative & Generic and by Delivery)—Global Industry Analysis, Size, Share, Growth, Trends and Forecast, 2012-2018. 2013. p. 128]. Despite this, we are not aware of any reports of a peptide that inhibits and/or prevents bacterial resistance to BZK and to other biocide mainstays of hospital and health care facilities. In this context, we examined the ability of the prototypic inhibitor peptides described herein to resensitize pathogenic bacteria to BZK and related disinfectants. FIG. 7 shows that addition of the peptide Ac-A-(Sar)₃-LLGIGLIIAGVLV-KKK-NH₂—The 88-100 TM4 sequence from Pseudomonas aeruginosa Psmr—in the presence of a sub-lethal concentration of BZK completely abolishes E. coli growth, i.e., the bacteria become susceptible to low concentrations of BZK. A ‘scrambled’ analog of the TM4 peptide—used as a control that eliminates the required peptide-protein interaction motif—was inactive in this experiment.

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1. A peptide comprising the sequence:
X₁-X₂-(G or S)-X₃-X₄-L-(I or M)-X₅-X₆-G-(V or I)-X₇X₈
wherein each of X₁-X₈ is independently any amino acid and is independently present or absent; wherein the peptide has fewer than 30 amino acid residues; and wherein the peptide binds to and inhibits an efflux pump, with the proviso that the peptide does not comprise VVGLALIVAGVVV or VVGLALINAGVVV.

2. The peptide of claim 2, wherein each of X₁-X₈ is present.

3. The peptide of claim 1, wherein:
X₁ is V, F, I, L, or C;
X₂ is V, I, or L;
X₃ is L, M, or I;
X₄ is A, G, I, M, V, or L;
X₅ is V, I, C, or G;
X₆ is A, S, V, F, C, or T;
X₇ is V, L, or I; and
X₈ is V, T, L, or I,
optionally wherein up to three of X₁-X₈ are independently replaced with a residue selected from N and Q so as to reduce the overall hydrophobicity of the peptide.

4. The peptide of claim 1, comprising a sequence selected from the group consisting of:
FVGMGLIVSGVVV;
VVGIGLIVVGVVT;
IISIILIIFGVVL;
LLGIGLIIAGVLV;
CIGLALMIAGIVI;
IIGMMLICAGVLV;
IVSIVLIIVGVVL;
IIGMLLIICGVIV;
IIGMMLICTGVLV; and
IIGIGLIIAGVVV.

5. The peptide of claim 1, further comprising a positively charged solubility-increasing tag, wherein the solubility-increasing tag comprises one or more positively charged amino acid residues, wherein the positively charged amino acid residues are selected from lysine and/or arginine residues, such as lysine residues, and/or an uncharged membrane-insertion tag, wherein the membrane-insertion tag comprises one or more peptoid residues, wherein the peptoid residues are selected from NVal (N-isopropylglycine), NLeu (N-isobutylglycine), and/or NAla (N-methylglycine; sarcosine), such as sarcosine.

6-8. (canceled)

9. The peptide of claim 5, wherein the solubility-increasing tag and/or the membrane-insertion tag is at the C- or N-terminus of the peptide, such as the N-terminus.

10-14. (canceled)

15. The peptide of claim 9, wherein the membrane-insertion tag is at the N-terminus and comprises an N-terminal amino group blocking moiety, such as an N-acetyl-Ala residue.

16. The peptide of claim 15, wherein the membrane-insertion tag comprises the sequence N-acetyl-Ala-Sar-Sar-Sar-.

17. The peptide of claim 9, wherein the membrane-insertion tag is at the C-terminus and comprises a C-terminal carboxylate group blocking moiety, such as a Sar-methyl ester residue.

18. The peptide of claim 17, wherein the membrane-insertion tag comprises the sequence -Sar-Sar-Sar-Sar-methyl ester.

19. The peptide of claim 1, wherein the peptide is stapled/macrocyclic.

20. The peptide of claim 19, wherein the peptide is stapled via or between residues X₄ and X₆.

21. The peptide of claim 1, wherein the peptide has a hydrophobicity above that required for insertion into a bilayer membrane but below that required for hemolysis of red blood cells.

22. The peptide of claim 1, wherein the efflux pump is a bacterial drug efflux pump and is a member of the SMR family.

23. (canceled)

24. The peptide of claim 1, wherein the peptide comprises 5, 6, 7, 8, 9, 10, 11, 12, 13, 14, 15, 16, 17, 18, 19, 20, 21, 22, 23, 24, 25, 26, 27, 28, 29, or 30 amino acid residues, such as 13 amino acid residues.

25. A peptide comprising the sequence IIGIGLIIAGVVV, or a fragment or variant thereof having at least 50%, identity to IIGIGLIIAGVVV, wherein the peptide has fewer than 30 amino acid residues and wherein the peptide binds to and inhibits a drug efflux pump, with the proviso that the peptide does not comprise VVGLALIVAGVVV or VVGLALINAGVVV.

26-45. (canceled)

46. A peptide comprising the sequence NQAPSLYAISLIVVFLCLAALYESWSI, or a fragment or variant thereof having at least 50%, 60%, 70%, 75%, 80%, 85%, 90%, 95%, 96%, 97%, 98%, or 99% identity to NQAPSLYAISLIVVFLCLAALYESWSI, wherein the peptide has fewer than 30 amino acid residues and wherein the peptide binds to and inhibits a drug efflux pump.

47. The peptide of claim 46, wherein the peptide binds to and inhibits interactions between TM1 and TM8 of the RND drug efflux pump.

48-94. (canceled)

95. A combination comprising an antibiotic and the peptide of claim 1.

96. The combination of claim 95, wherein the combination synergistically treats an infection.

97. The combination of claim 95, wherein the antibiotic and peptide are in the same composition.

98-102. (canceled)
